(19) United States
(12) Patent Application Publication
(10) Pub. No.: US 2012/0101390 A1
(43) Pub. Date: Apr. 26, 2012

(54) Multi-Modal Imaging for Diagnosis of Early Stage Epithelial Cancers

(76) Inventors: Nicusor V. Iftimia, Chelmsford, MA (US); Niyom Lue, Nahant, MA (US); Daniel X. Hammer, Bedford, NH (US); Mircea Mujat, Acton, MA (US); R. Daniel Ferguson, Melrose, MA (US)

(21) Appl. No.: 13/277,481

(22) Filed: Oct. 20, 2011

Related U.S. Application Data
(60) Provisional application No. 61/405,059, filed on Oct. 20, 2010.

(51) Int. Cl. A61B 6/00; A61K 49/00; B82Y 15/00

(52) U.S. Cl. 600/476; 424/9.6; 977/773; 977/927

(57) ABSTRACT

Epithelial cancer screening can include a staining tissue with a cancer targeting agent, identifying a potentially cancerous lesion using fluorescence imaging, and imaging the potentially cancerous lesion for a cancer diagnosis using optical coherence tomography.

### CROSS-REFERENCE TO RELATED APPLICATION

This application claims the benefit of and priority to U.S. Provisional Patent Application No. 61/405,059 filed Oct. 20, 2010, which is owned by the assignee of the instant application and the disclosure of which is incorporated herein by reference in its entirety.

### GOVERNMENT RIGHTS

The invention was made with government support from the National Institute of Health under grant number 5R41 CA 132256-02. The government may have certain rights in the invention.

### FIELD OF THE INVENTION

The invention relates generally to endoscopic diagnosis of early stage epithelial cancers, and more particularly to cancer screening using a multi-modal imaging system and a cancer targeting molecular probe.

### BACKGROUND

Traditional imaging modalities, such as computed invention (CT) scans, magnetic resonance (MR), and endoscopy can be used for cancer screening. Due to lack of resolution, they can only detect lesions that are already formed, which are more difficult to treat. For example, colonoscopy can only detect adenomatous polyps, from which cancer usually arises. However, detecting cancer in its very early stage, when it is confined within the epithelial layer of the organ (stages I and II), can result in the cancer being treatable and even curable with surgery. If cancer is detected in latter stages, when it already spreads to regional lymph nodes (stage III), the cure rate degrades significantly.

### SUMMARY OF THE INVENTION

While Optical Coherence Tomography (OCT) can be optimized as a diagnostic tool for detection and characterization of epithelial cancer, it typically has a limited field of view, on the order of millimeters, and thus scanning of large organs is not very practical because it involves the time-consuming analysis of very large data sets. In addition, it can substantially increase the length of the procedure. The use of a guidance tool, like fluorescence imaging, can help to highlight the presence of cancer-suspect lesions. Thus, OCT imaging can be performed on these highlighted areas to visualize tissue morphology and confirm or rule out cancer presence.

While endogenous and exogenous fluorescence imaging has shown to be relatively sensitive to cancer, its specificity can be improved with the use of a molecular markers (e.g., peptides, antibodies, etc.) that are cancer specific. Nanoparticles or microparticles can be functionalized with markers and loaded with a fluorophore to enhance the contrast of fluorescence imaging. In this way, early stage epithelial cancers can be detected, which can be otherwise undetectable with current endoscopic approaches. Early stage epithelial cancer diagnosis can be improved by targeting the cancer cells with a contrast agent that recognizes the specific receptors on the cell and by visualizing the targeted area of the tissue by using a multimodal endoscopic approach: combined bright field-fluorescence-optical coherence tomography imaging. In addition to bright field color images, this new approach can provide a clinician with enhanced contrast fluorescence images, which can highlight the location of early stage lesions, and with high-resolution cross-sectional OCT images in these highlighted areas. This approach can also significantly improve the diagnostic yield of current endoscopy procedures.

Fluorescence contrast enhancement can be provided by a cancer-targeting agent that can be topically delivered. The agent (e.g., the nanoparticles or microparticles) can bind to cancer cells and fluoresce when excited with near infrared (NIR) light. Biopsy can still be performed to confirm/rule out cancer presence. However, the number of biopsies can be minimized by performing them only on the suspicious areas indicated by combined fluorescence/OCT imaging

The invention, in one embodiment, features a multimodal imaging invention for more efficient diagnosis of early stage epithelial cancers. Organs that can be screened include, but are not limited to, the colon, bladder, cervix, oral cavity, and esophagus. A combined, enhanced contrast fluorescence/OCT imaging system can enhance the findings of the regular endoscopy procedures by providing a clinician with additional high-contrast fluorescence images of the tissue, highlighting suspicious areas, as well as with high-resolution cross-sectional OCT images showing the true morphology of the tissue in the fluorescently highlighted regions. To highlight cancer presence, a cancer targeting agent loaded with a fluorophore is topically applied. This agent can recognize one or more cancer receptors. For example, the agent can be based on argenine-glycine-aspartic acid (RGD)-functionalized gold nanoparticles, which recognize either the αᵥβ₃ integrin receptor, (ABIR), or the epidermal growth factor (EGF) receptor, or both, which are over-expressed by epithelial cancer cells. Users of the system can include health care providers where endoscopies are carried out, including medical centers, regional hospitals, local clinics and physician practice groups that perform endoscopies on an out-patient basis.

A combined, enhanced contrast fluorescence/OCT imaging system can be more reliable, sensitive and specific than conventional technologies used to detect cancer in its very early stage. This approach and imaging system can provide the net advantage that it minimizes drug spreading in other organs, since the contrast agent is applied topically, and therefore significantly reduces the overall body toxicity effects. The imaging system can provide true images of tissue architecture at a scale very close to that of histopathology, can provide faster diagnostic results obtained while a patient waits, thus decreasing patient anxiety and accelerating commencement of any treatment needed, and can provide an alternative and safer option to virtual colonoscopy (CT scans).

In one aspect, there is a method for epithelial cancer screening. The method includes staining tissue with a cancer targeting agent, identifying a potentially cancerous lesion using fluorescence imaging, and imaging the potentially cancerous lesion for a cancer diagnosis using optical coherence tomography.

In another aspect, there is a method for epithelial cancer screening. The method includes staining tissue with a cancer targeting agent, and delivering, using illumination fibers of an endoscope, first source radiation to the tissue for fluorescence imaging. Fluorescence radiation emitted by a dye of the cancer targeting agent in the tissue is received using a first channel of the endoscope. A potentially cancerous lesion is identified based on a fluorescence image of the tissue. Second source radiation is delivered, using a second channel of the endoscope, to the tissue including the potentially cancerous lesion for optical coherence tomography (OCT) imaging. Radiation emitted by the tissue including the potentially cancerous lesion is received using the second channel of the endoscope to form an OCT image, which is analyzed to determine a cancer diagnosis for the potentially cancerous lesion. In certain embodiments, third source radiation is delivered to the tissue for bright field imaging using the illumination fibers of the endoscope. Second radiation emitted by the tissue is received using the first channel of the endoscope to form a bright field image of the tissue. The bright field image and the fluorescence image are co-registered to identify the potentially cancerous lesion.

In still another aspect, there is a method for epithelial cancer screening. The method includes delivering, using a first surface of an optic of a handheld probe, first source radiation to tissue stained with a cancer targeting agent for fluorescence imaging, and receiving, using the first surface of the optic of the handheld probe, fluorescence radiation emitted by a dye of the cancer targeting agent in the tissue. The fluorescence radiation is directed to a first detector of the handheld probe to acquire a fluorescence image of the tissue, and a potentially cancerous lesion is identified based on the fluorescence image of the tissue. OCT imaging radiation is raster scanned on the tissue including the potentially cancerous lesion through a second surface of the optic of the handheld probe. Radiation emitted by the tissue including the potentially cancerous lesion is received through the first surface of the optic of the handheld probe. The radiation emitted is directed to a second detector to acquire an OCT image of the tissue, which is analyzed to determine a cancer diagnosis for the potentially cancerous lesion. In certain embodiments, third source radiation is delivered to the tissue for bright field imaging using the first surface of the optic of the handheld probe. Second radiation emitted by the tissue is received using the first surface of the optic of the handheld probe and directed to a third detector of the handheld probe to acquire a bright field image of the tissue. The bright field image and the fluorescence image are co-registered to identify the potentially cancerous lesion.

In another aspect, there is a multimodality imaging system for screening for epithelial cancer. The system includes an endoscope defining a first channel for fluorescence imaging and a second channel for optical coherence tomography (OCT) imaging. The endoscope includes illumination fibers running coaxially with the first channel and the second channel. The illumination fibers are coupled to a first source of radiation for the fluorescence imaging. A distal end of the first channel is configured to receive fluorescence radiation emitted by a dye of the cancer targeting agent and direct the fluorescence radiation to a first detector to acquire a fluorescence image of the tissue. The second channel is coupled to a second source for OCT imaging radiation. A distal end of the second channel is configured to receive OCT radiation emitted by the tissue including a potentially cancerous lesion and direct the OCT radiation emitted to a second detector to acquire an OCT image of the tissue. A processor is coupled to the first detector and the second detector. The processor is configured to identify the potentially cancerous lesion using the fluorescence imaging and image the potentially cancerous lesion for a cancer diagnosis of the tissue. The endoscope can include a third channel for a catheter configured to deliver the cancer targeting agent. In certain embodiments, the illumination fibers are coupled to a third source of radiation for bright field imaging. The distal end of the first channel is configured to receive radiation emitted by the tissue and direct the radiation to a third detector to acquire a bright field image of the tissue. The processor is coupled to the third detector and is configured to co-register the bright field image and the fluorescence image of the tissue to identify the potentially cancerous lesion.

In still another aspect, there is a multimodality imaging system for screening for epithelial cancer. The system includes a hand held probe including a first optic having a first surface configured (i) to direct first source radiation to tissue stained with a cancer targeting agent for fluorescence imaging, (ii) receive fluorescence radiation emitted by a dye of the cancer targeting agent in the tissue, and (iii) direct the fluorescence radiation to a first detector of the handheld probe to acquire a fluorescence image of the tissue. A system of optics is configured to raster scan, through a second surface of the first optic, OCT imaging radiation on the tissue including a potentially cancerous lesion, and direct OCT radiation emitted by the tissue to a second detector to acquire an OCT image of the tissue. A processor is coupled to the first detector and the second detector. The processor is configured to identify the potentially cancerous lesion using the fluorescence imaging and image the potentially cancerous lesion for a cancer diagnosis of the tissue. In certain embodiments, the first optic has a first surface is configured to direct third source radiation to tissue for bright field imaging, receive radiation emitted by the tissue, and direct the radiation to a third detector to acquire a bright field image of the tissue. The processor is coupled to the third detector and is configured to co-register the bright field image and the fluorescence image of the tissue to identify the potentially cancerous lesion.

In still another aspect, there is kit for epithelial cancer screening. The kit includes a cancer targeting agent, a probe including a first channel for combined bright field/fluorescence imaging and a second channel for OCT imaging, and instruction means. The instruction means includes instructions for staining tissue with the cancer targeting agent, identifying a potentially cancerous lesion using the fluorescence imaging, and imaging the potentially cancerous lesion for a cancer diagnosis using the OCT imaging.

In yet another aspect, there is a system for epithelial cancer screening. The system includes means for staining tissue with a cancer targeting agent, means for identifying a potentially cancerous lesion, and means for imaging the potentially cancerous lesion for a cancer diagnosis. The means for identifying can be fluorescence imaging. The means for diagnosis can be optical coherence tomography.

In other examples, any of the aspects above, or any apparatus, system or device, or method, process or technique, described herein, can include one or more of the following features. In certain embodiments, endoscopic fluorescence guided optical coherence tomography imaging is utilized. A hand held probe can be used for oral cancer screening. An endoscopic probe can be used for not directly accessible organs.

The cancer targeting agent can have high specificity. The agent can be based on argenine-glycine-aspartic acid functionalized gold nanoparticles or microparticles. The agent can be adapted to recognize the αᵥβ₃ integrin and/or epidermal growth factor (EGF) receptors. The cancer targeting agent can be based on gold colloids adsorbed poly(epsilon-caprolactone) (Au-PCL) microparticles labeled with a near-infrared (NIR) dye and functionalized with an RGD peptide. The cancer targeting agent can be topically delivered to the tissue using, e.g., a catheter or agent delivery system.

Other aspects and advantages of the invention will become apparent from the following detailed description, taken in conjunction with the accompanying drawings, illustrating the principles of the invention by way of example only.

### BRIEF DESCRIPTION OF THE DRAWINGS

The advantages of the invention described above, together with further advantages, may be better understood by referring to the following description taken in conjunction with the accompanying drawings. The drawings are not necessarily to scale, emphasis instead generally being placed upon illustrating the principles of the invention.

FIG. 1 shows an exemplary probe for fluorescence imaging and OCT imaging.

FIG. 2A shows a schematic of a multimodality optical system for endoscopic implementation.

FIG. 2B shows a sectional view of the distal end of an endoscope.

FIG. 3 shows an example of an image console.

FIG. 4A shows a bright field image of a portion of a colon including fluorescence showing potentially cancerous lesions.

FIG. 4B shows an OCT image of a fiducial line of the colon shown in FIG. 4A.

FIG. 5 shows a schematic diagram of a multimodality optical system for hand-held implementation.

### DETAILED DESCRIPTION OF THE INVENTION

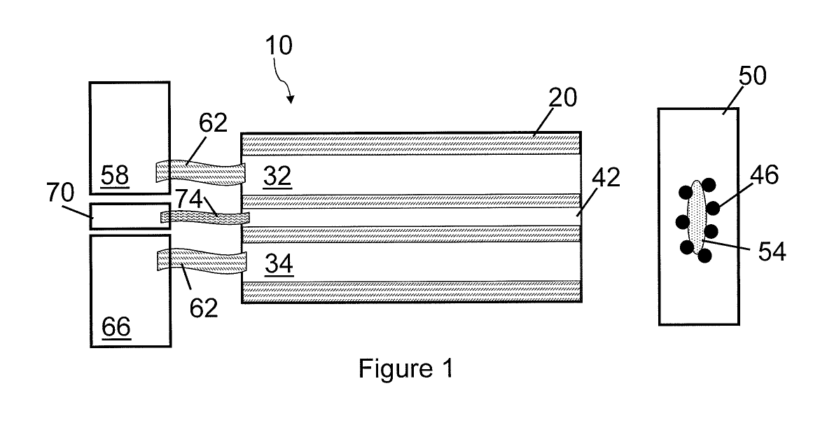

FIG. 1 shows an exemplary probe 10 including a body 20 defining a first channel 32 for fluorescence imaging and a second channel 34 for OCT imaging. The first channel 32 can be used for bright field imaging, or a separate channel can be used for bright field imaging. The endoscopic probe 10 can define a third channel 42 used for delivery of a cancer targeting agent 46, such as the RGD-functionalized Au-PCL microparticles or nanoparticles, to tissue 50. The cancer targeting agent 46 can stain the tissue 50 containing a potentially cancerous lesion 54. Fluorescence imaging is used to identify the potentially cancerous lesion, and OCT imaging of the potentially cancerous lesion is used for cancer diagnosis. The endoscopic probe 10 need not include the third channel 42. In certain embodiments, the cancer targeting agent 46 can be introduced through alternative means, such as direct delivery or by a catheter.

The first channel 32 can be coupled to a fluorescence imaging system 58 by an optical waveguide 62, e.g., one or more optical fibers, optical fiber bundles, or light guides. The second channel 34 can be coupled to an OCT imaging system 66 by an optical waveguide 62. Each of the first and second channels can be an optical waveguide, or the channel can be a bore through the endoscopic probe 30 into which an optical waveguide is insertable. The third channel 42 can be coupled to a delivery system 70 by tubing 74, e.g., a PTFE tubing. The third channel 42 can be tubing, or the third channel can be a bore through the endoscopic probe 10 into which tubing is insertable. The delivery system 70 can include a reservoir for the cancer targeting agent 46 or the reservoir can be a portion of the probe 30. The delivery system 70 can include a pump or syringe for directing the cancer targeting agent 46 to the tissue 50. In certain embodiments, the delivery system 70 is not a portion of the endoscopic probe 10. Instead, the cancer targeting agent 46 can be delivered by a delivery system 70 separate from the endoscopic probe 10. The delivery system 70 can be a needle or syringe that delivers the cancer targeting agent 46 to the tissue directly or via tubing. Each channel can be a tubular passage through the probe and/or a route for communication of radiation to and from the tissue or of the agent to the tissue.

The cancer targeting agent 46 can bind to EGF, integrin, or other cancer receptors with high sensitivity and specificity. The cancer targeting agent 46 is nontoxic or can provide negligible toxicity. The cancer targeting agent 46 can fluoresce in the near-infrared to minimize the autofluorescence contribution of some endogenous absorbers such as hemoglobin, water, and lipids.

In addition, near-infrared fluorescence imaging has provided an effective solution for improving the imaging depth along with sensitivity and specificity. Therefore, fluorophores emitting in the NIR region can assist in vivo optical imaging and future clinical applications. Among them, the NIR fluorescent fluorophores most commonly used for in vivo imaging are some synthetic cyanine analogs such as commercially available DiR 800, Cy7, Cy7.5, and their analogs of different conjugation groups and linkers.

To minimize the dye toxicity, the dye can be incorporate into a biodegradable polycaprolactane (PCL) matrix. The dye can be placed into the core of a PCL particle. In addition, to avoid systemic contamination, the size of the particle can be selected to be on the micron level, so that the retention into the leaky tumor vasculature is minimized.

With regard to the binding properties of the contrast agent, different receptors can be targeted, function of the cancer and screened organ. For colon cancers, integrin receptors are commonly used. Among the known 24 integrin subtypes, integrin αᵥβ₃ has been widely investigated for tumor imaging and therapy due to its important roles in angiogenesis, growth, and metastasis of some tumors. Integrin αᵥβ₃ binds some extracellular matrix proteins such as vitronectin via Arg-Gly-Asp (RGD) sequence, so diverse RGD peptide ligands have been developed to target integrin αᵥβ₃ and angiogenic vessels.

The cancer targeting agent can have high specificity. The agent can be based on argenine-glycine-aspartic acid functionalized gold nanoparticles or microparticles. The agent can be adapted to recognize the αᵥβ₃ integrin and/or epidermal growth factor (EGF) receptors. The cancer targeting agent can be based on gold colloids adsorbed poly(epsilon-caprolactone) (Au-PCL) microparticles labeled with a near-infrared (NIR) dye and functionalized with an RGD peptide. The gold nanoparticles can have a diameter of about 10 to about 30 nanometers (e.g., about 20 nanometers). The nanoparticle surfaces can be functionalized with argenine-glycine-aspartic acid (RGD) with a linear sequence ACD/CRG/DCF/CGG/GGG/COOH. The peptide can recognize the αᵥβ₃ integrin receptor overexpressed by the pre-cancerous epithelial cells.

A monomeric RGD has provided promising results both in vitro and in vivo, but its moderate integrin binding affinity may weaken tumor uptake and related in vivo performance. Therefore, dimerization or multimerization of the cyclic RGD peptide can improve the binding affinity and in vivo imaging. For example, various dimeric and tetrameric RGD analogues including E-[c(RGDfK)]2, E-[G-G-G-c(RGDfK)]2 and E-{G-[c(RGDfK)]2}2 can be used because they exhibit stronger binding than the monomeric counterpart in integrin αᵥβ₃-positive U87MG xenograft models.

Suitable cancer targeting agents are described in Cheng et al., “Near-infrared fluorescent RGD peptides for optical imaging of integrin αvβ3 expression in living mice.” Bioconjug Chem. 16:1433-41 (2005); Liu et al. “Optical imaging of integrin αvβ3 expression with near-infrared fluorescent RGD dimer with tetra(ethylene glycol) linkers,” Mol Imaging. 9:21-9 (2010); and Lue et al., “Preliminary evaluation of a nanotechnology-based approach for the more effective diagnosis of colon cancers,” Nanomedicine 5(9):1467-1479 (2010), the entire contents of all of which are hereby incorporated by reference in their entireties.

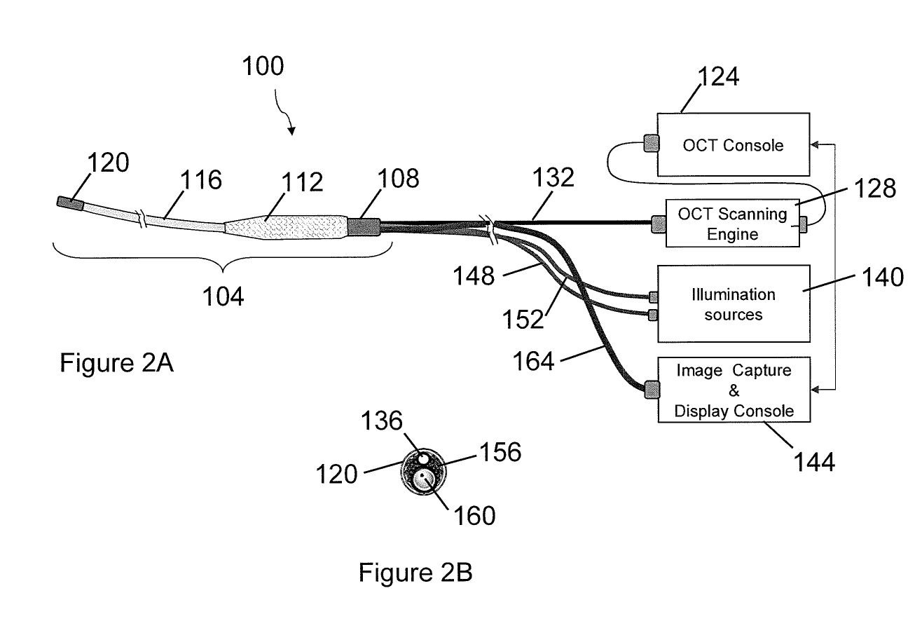

FIGS. 2A and 2B show an endoscopic implementation 100 of the multimodal imaging approach. An endoscope 104, which can be commercially available, includes an input port 108, a handle 112, a carrier tube 116 (which can be flexible), and a distal end 120. The endoscope 104 includes a first channel for the fluorescence and/or brightfield imaging system and a second channel for the OCT imaging system. An OCT scanning engine 128 can be coupled to an OCT console 124, and an OCT catheter 132 can be attached. As shown in FIG. 2B, the OCT channel terminates at an OCT port 136, from which radiation is directed to the tissue and by which the radiation returning from the tissue is received.

The OCT imaging system can be configured in a spectrometer based architecture, although a swept source-based architecture can be used. Time domain OCT can also be used. The OCT console can include an aiming source operate at about 835 nm and an OCT source operating at about 1310 nm. The detector can be a linear array or digital camera (e.g., a digital InGaAs camera).

The endoscopic implementation 100 includes one or more illumination sources 140 and an image capture and display console 144. The illumination sources 140 can be used for fluorescence imaging and/or brightfield imaging. In FIG. 2, a first illumination fiber 148 can be for the fluorescence excitation radiation and a second illumination fiber 152 can be for the brightfield excitation radiation. The illumination fibers can include one or more optical fibers or can be a fiber bundle. FIG. 2B shows the illumination fibers 148 and 152 in section view (referred to by reference number 156 in FIG. 2B).

Imaging port 160 can be adapted to allow for co-registered bright-field/fluorescence imaging. Imaging port 160 is coupled to imaging bundle 164, which is coupled to the image capture and display console 144. Imaging port 160 can terminate with a micro-objective lens, which can collect the radiation returning from the tissue.

The illumination source 140 can be a laser, a high power LED or an incoherent source. In certain embodiments, the illumination source is a combination of a laser with a high power LED or an incoherent source. Suitable lasers include diode lasers, such as a 745 nm laser diode. Suitable incoherent sources include halogen lamps or arc lamps such as Xenon arc lamps. The illumination source can excite the NIR dye. The source can provide continuous wide band of light used as the illumination source for the bright field imaging and/or fluorescence excitation. A halogen lamp can be used as an incoherent illumination source for bright field imaging.

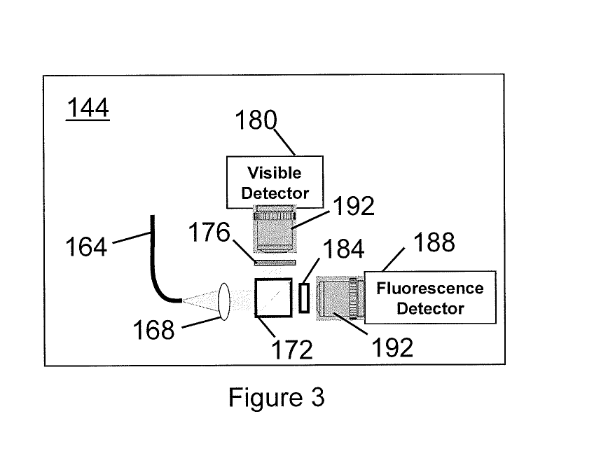

FIG. 3 shows an example of image console 144, which can separate the visible and fluorescence images. Imaging bundle 164 delivers radiation to an objective lens 168, which directs radiation to a beamsplitter 172 (e.g., a pellicle beamsplitter or a dichroic beamsplitter). A dichroic beamsplitter can be used for efficient management of the detected photons. A first filter 176 blocks the laser source from entering the visible detector 180, while a second filter 184 blocks visible radiation from entering the fluorescence detector 188. Block filters can prevent background interference in either of the two imaging channels. Each detector includes a respective objective 192.

The fluorescence detector 188 can be a high sensitivity CCD camera (Rolera XR, Q-Imaging BC, Canada) or a photomultiplier tube (PMT). The filter 184 can block back reflection signal from the sample and allow only the emission from the fluorescence dye to reach the CCD sensor.

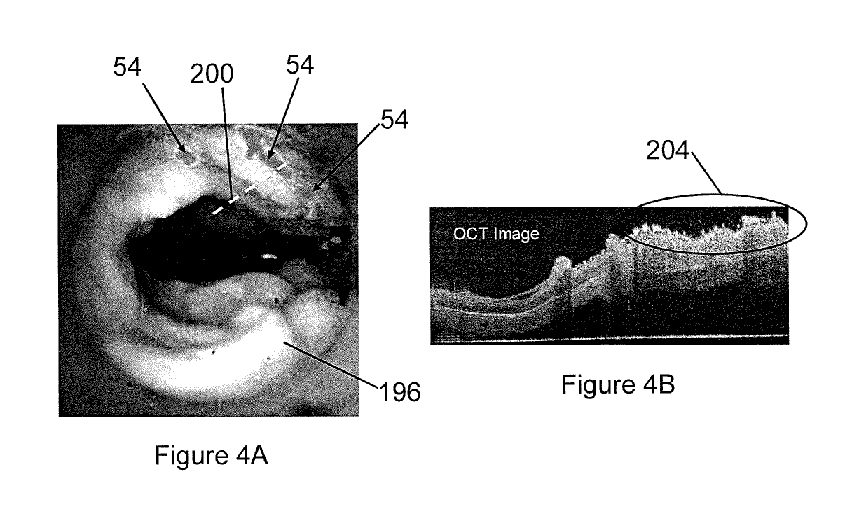

Image console 144 includes software to co-register the bright-field and fluorescence images and show a fiducial line of the OCT beam. FIG. 4A shows a bright field image of a portion of a colon 196. A potentially cancerous lesion 54, shown as dark patches in the image, is fluorescing. FIG. 4B shows an OCT image along a fiducial line of the colon, shown as a dashed white line 200 in FIG. 4A. Potentially cancerous lesion 54 is circled 204 in FIG. 4B.

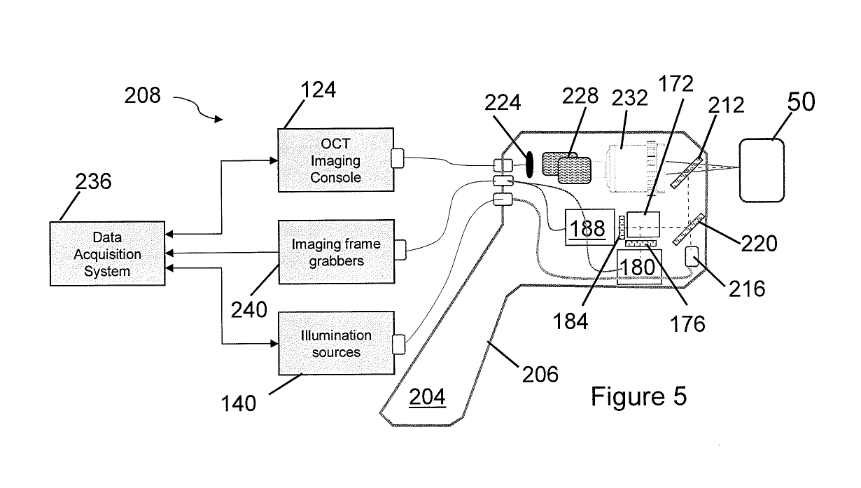

FIG. 5 shows a hand-held multimodality implementation 204 of an optical system 208 for oral cancer diagnosis. The optical system 208 includes a fluorescence imaging system and an OCT imaging system coupled to the handheld probe 204. The probe 204 is used to deliver radiation to and receive radiation from tissue sample 50. The probe 204 includes a dichroic beam splitter 212 to couple radiation from the fluorescence imaging system and radiation from the OCT imaging system to the tissue 50.

The probe 204 includes a handle 206. The optical system 208 includes illumination source 140, beamsplitter 172, first filter 176, visible detector 180, second filter 184, fluorescence detector 188, and an objective 192. Illumination source 140, which is external to the handpiece 204, is fiber coupled to an emitter 216 inside the handpiece 204.

Dichroic beam splitter 212 can have a first surface that directs radiation from the emitter 216 to the tissue 50 and allows OCT radiation from the OCT console 124 to pass to the tissue 50. Fluorescence radiation and OCT radiation returning from the tissue 50 is redirected toward the detectors. Dichroic beam splitter 220 can allow radiation from emitter 216 to pass, but redirects radiation returning from the tissue 50 to the detectors. The OCT imaging system includes an OCT imaging console 124, a fiber-pigtailed collimating lens 224, two scanning mirrors 228 that generate an OCT raster scan and a scanning lens 232 (e.g., a 5× scanning lens (Model LSM03, Thorlabs, N.J., USA)). The imaging probe 204 can collect raster OCT images with an axial resolution of about 5 to 10 μm in tissue and relatively large field fluorescence images at relatively high frame rates (20 fps or higher, depending of the image resolution).

A data acquisition system 236 can include a computer processor, a data acquisition board, and a display, and be coupled to frame grabbers 240. The data acquisition system 236 can be used to control the fluorescence imaging system and the OCT imaging system, and collect data from while imaging. The data acquisition system 236 can be used to combine the OCT and fluorescence excitation beams on the tissue sample and simultaneously collect both OCT and fluorescence images for identification of potentially cancerous lesions and cancer diagnosis. Frame grabbers 240 receive imaging information from detectors 180 and 188.

A mouse study was used to demonstrate the preferential binding of the contrast agent (e.g., AuNP-coated PCL microparticles) to the tumor tissue and the capability of fluorescence imaging to highlight cancer presence. 10 adult outbred nu/nu mice were used in this study. Approximately, 4×10⁶ HT-29 cells suspended in 100 μl of media (1:1 ratio of serum supplemented DMEM and matrigel HC) were injected subcutaneously into the dorsal side of female nu/nu mice under light isoflurane anesthesia. Subcutaneous colon cancer tumors developed in 2-3 weeks. As soon as tumor volume reached about 500 mm³, mice were sacrificed and tumors were excised. Normal tissue surrounding the tumor was excised as well. Tissue was cut in 50 μm thickness slices for microscope imaging and in 2 mm slices for OCT/Fluorescence imaging. At least 2 thick slices and 3-4 thin slices of each tissue type were obtained for each mouse. The tissue slices were incubated for 2 hrs with the PCL-DiR-Au-RGD compound (2 mg microparticles/50 ml of PBS), then washed 3-5 times intensively with PBS, and kept in saline solution at 37° C. during imaging.

Bright field and fluorescence images were collected and analyzed with ImageJ software. Few particles attached to the normal tissue, while the density of the particles on the tumor tissue is orders of magnitude higher.

A statistical analysis of microparticle binding to mouse tissue was performed on over 20 microscope slides. The microparticle density on tumor slides was almost two orders of magnitude higher than the density on normal tissue slides. Particle counting was performed on the brightfield images over areas of 30×25 μm² using ImageJ software. Again, this demonstrates the preferential attachment of the functionalized microparticles to tumor tissue.

Two tissue slices per animal (one normal and one tumor) were analyzed with the OCT/fluorescence imaging setup. Besides the 1310 nm beam, an 830 nm aiming beam was launched into the OCT channel of the imaging probe to evidence the position of the OCT scan on the fluorescence image. An offset signal was software generated for the OCT galvanometers to move the OCT scan in any region-of-interest (ROI) in the fluorescence image.

For the same exposure time of the camera (2.5 ms), the tumor tissue is significantly brighter than the normal tissue due to the higher density of microparticles. While performing OCT imaging, significant differences were observed between the highly and lower fluorescing tissues. While the lower fluorescing tissue showed a uniform decrease of the signal intensity with imaging depth, which is typical to normal fibrotic tissues, the highly fluorescing tissue showed a very irregular structure, which is specific to tumor tissues.

A human colon tissue study was performed also. Tumor and normal colon tissue samples (5 of each tissue-type) were procured from the National Disease Research Interchange (NDRI) and were incubated for 2 hours with the microparticle formulation (2 mg microparticles/50 ml PBS). The microparticle solution was topically applied on the colon tissue. After 2 hours of incubation, the colon samples were washed 3-5 times with PBS and placed in a saline solution. OCT and fluorescence images were taken on both normal and carcinogenic tissue samples before and after microparticle incubation. The fluorescence imaging showed increased brightness on the cancer tissue sample compared to the normal tissue sample, demonstrating the preferential binding of the microparticles to the cancer sample, while the OCT imaging showed a clear difference between normal and tumor tissue.

The microparticles can provide some degree of contrast enhancement in the OCT images of the cancer tissue. An OCT image of the cancer tissue after microparticle incubation shows an increased brightness over a depth of approximately 250 μm. This might correspond to microparticles attachment to the several top layers of cells. Due to their relatively large size, the microparticles may not be able to go deeper into the very dense cancer tissue. However, the OCT images of the normal tissue look very similar before and after microparticle incubation. This demonstrates that the microparticles did not stain the normal tissue.

The invention features a kit suitable for endoscopic diagnosis of early stage epithelial cancers. The kit can include a cancer targeting agent, an endoscope, and instruction means. The endoscope can include a first channel for fluorescence imaging and a second channel for high-resolution OCT imaging. The cancer targeting agent can be delivered to tissue using a delivery system for a cancer targeting agent. The instruction means can include instructions staining the tissue with the cancer targeting agent, identifying a potentially cancerous lesion using fluorescence imaging, and imaging the potentially cancerous lesion for a cancer diagnosis using optical coherence tomography. The instruction means, e.g., treatment guidelines, can be provided in paper form, for example, as a leaflet, booklet, book, manual, or other like, or in electronic form, e.g., as a file recorded on a computer readable medium such as a drive, CD-ROM, DVD, or the like.

The above-described techniques and/or the instruction means can be implemented in digital electronic circuitry, or in computer hardware, firmware, software, or in combinations of them. The implementation can be as a computer program product, i.e., a computer program tangibly embodied in an information carrier, e.g., in a machine-readable storage device or in a propagated signal, for execution by, or to control the operation of, data processing apparatus, e.g., a programmable processor, a computer, or multiple computers. A computer program can be written in any form of programming language, including compiled or interpreted languages, and it can be deployed in any form, including as a stand-alone program or as a module, component, subroutine, or other unit suitable for use in a computing environment. A computer program can be deployed to be executed on one computer or on multiple computers at one site or distributed across multiple sites and interconnected by a communication network.

Method steps can be performed by one or more programmable processors executing a computer program to perform functions of the technology by operating on input data and generating output. Method steps can also be performed by, and apparatus can be implemented as, special purpose logic circuitry, e.g., a FPGA (field programmable gate array), a FPAA (field-programmable analog array), a CPLD (complex programmable logic device), a PSoC (Programmable System-on-Chip), ASIP (application-specific instruction-set processor), or an ASIC (application-specific integrated circuit), or the like. Subroutines can refer to portions of the stored computer program and/or the processor, and/or the special circuitry that implement one or more functions.

Processors suitable for the execution of a computer program include, by way of example, both general and special purpose microprocessors, and any one or more processors of any kind of digital computer. Generally, a processor can receive instructions and data from a read-only memory or a random access memory or both. The essential elements of a computer are a processor for executing instructions and one or more memory devices for storing instructions and data. Generally, a computer can also include, or be operatively coupled to receive data from or transfer data to, or both, one or more mass storage devices for storing data, e.g., magnetic, magneto-optical disks, or optical disks. Data transmission and instructions can also occur over a communications network. Information carriers suitable for embodying computer program instructions and data include all forms of non-volatile memory, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in special purpose logic circuitry.

The terms “module” and “function,” as used herein, mean, but are not limited to, a software or hardware component which performs certain tasks. A module may advantageously be configured to reside on addressable storage medium and configured to execute on one or more processors. A module may be fully or partially implemented with a general purpose integrated circuit (IC), DSP, FPGA or ASIC. Thus, a module may include, by way of example, components, such as software components, object-oriented software components, class components and task components, processes, functions, attributes, procedures, subroutines, segments of program code, drivers, firmware, microcode, circuitry, data, databases, data structures, tables, arrays, and variables. The functionality provided for in the components and modules may be combined into fewer components and modules or further separated into additional components and modules. Additionally, the components and modules may advantageously be implemented on many different platforms, including computers, computer servers, data communications infrastructure equipment such as application-enabled switches or routers, or telecommunications infrastructure equipment, such as public or private telephone switches or private branch exchanges (PBX). In any of these cases, implementation may be achieved either by writing applications that are native to the chosen platform, or by interfacing the platform to one or more external application engines.

To provide for interaction with a user, the above described techniques can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer (e.g., interact with a user interface element). Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input.

The above described techniques can be implemented in a distributed computing system that includes a back-end component, e.g., as a data server, and/or a middleware component, e.g., an application server, and/or a front-end component, e.g., a client computer having a graphical user interface and/or a Web browser through which a user can interact with an example implementation, or any combination of such back-end, middleware, or front-end components. The components of the system can be interconnected by any form or medium of digital data communication, e.g., a communication network. Examples of communication networks include a local area network (“LAN”) and a wide area network (“WAN”), e.g., the Internet, and include both wired and wireless networks. Communication networks can also all or a portion of the PSTN, for example, a portion owned by a specific carrier.

The computing system can include clients and servers. A client and server are generally remote from each other and typically interact through a communication network. The relationship of client and server arises by virtue of computer programs running on the respective computers and having a client-server relationship to each other.

While the invention has been particularly shown and described with reference to specific illustrative embodiments, it should be understood that various changes in form and detail may be made without departing from the spirit and scope of the invention.

What is claimed is:

1. A method for epithelial cancer screening, comprising:
staining tissue with a cancer targeting agent;
identifying a potentially cancerous lesion using fluorescence imaging; and
imaging the potentially cancerous lesion for a cancer diagnosis using optical coherence tomography.

2. The method of claim 1 further comprising topically staining the tissue using a delivery catheter.

3. The method of claim 1 wherein the method utilizes endoscopic fluorescence guided optical coherence tomography imaging.

4. The method of claim 1 wherein the cancer targeting agent can be based on gold colloids adsorbed poly(epsilon-caprolactone) (Au-PCL) microparticles labeled with a near-infrared (NIR) dye and functionalized with an RGD peptide.

5. The method of claim 1 wherein the agent is based on argenine-glycine-aspartic acid functionalized gold nanoparticles.

6. The method of claim 1 wherein the agent is adapted to recognize αᵥβ₃ integrin receptors.

7. The method of claim 1 wherein the agent is adapted to recognize EGF receptors.

8. A method for epithelial cancer screening, comprising:
staining tissue with a cancer targeting agent;
delivering, using illumination fibers of an endoscope, first source radiation to the tissue for fluorescence imaging;
receiving, using a first channel of the endoscope, fluorescence radiation emitted by a dye of the cancer targeting agent in the tissue;
identifying, based on a fluorescence image of the tissue, a potentially cancerous lesion;
delivering, using a second channel of the endoscope, second source radiation to the tissue including the potentially cancerous lesion for optical coherence tomography (OCT) imaging;
receiving, using the second channel of the endoscope, radiation emitted by the tissue including the potentially cancerous lesion to form an OCT image; and
analyzing the OCT image to determine a cancer diagnosis for the potentially cancerous lesion.

9. The method of claim 8 further comprising topically staining the tissue using a delivery catheter.

10. The method of claim 8 further comprising:
delivering, using the illumination fibers of the endoscope, third source radiation to the tissue for bright field imaging;
receiving, using the first channel of the endoscope, second radiation emitted by the tissue to form a bright field image of the tissue; and
co-registering the bright field image and the fluorescence image of the tissue to identify the potentially cancerous lesion.

11. A method for epithelial cancer screening, comprising:
delivering, using a first surface of an optic of a handheld probe, first source radiation to tissue stained with a cancer targeting agent for fluorescence imaging;
receiving, using the first surface of the optic of the handheld probe, fluorescence radiation emitted by a dye of the cancer targeting agent in the tissue;
directing the fluorescence radiation to a first detector of the handheld probe to acquire a fluorescence image of the tissue;
identifying, based on the fluorescence image of the tissue, a potentially cancerous lesion;
raster scanning, through a second surface of the optic of the handheld probe, OCT imaging radiation on the tissue including the potentially cancerous lesion;
receiving, through the first surface of the optic of the handheld probe, radiation emitted by the tissue including the potentially cancerous lesion;
directing the radiation emitted to a second detector to acquire an OCT image of the tissue;
analyzing the OCT image to determine a cancer diagnosis for the potentially cancerous lesion.

12. The method of claim 11 further comprising topically staining the tissue using a delivery catheter.

13. The method of claim 11 further comprising:
delivering, using the first surface of the optic of the handheld probe, third source radiation to the tissue stained with the cancer targeting agent for bright field imaging;
receiving, using the first surface of the optic of the handheld probe, second radiation emitted by the tissue;
directing the second radiation emitted to a third detector of the handheld probe to acquire a bright field image of the tissue; and
co-registering the bright field image and the fluorescence image of the tissue to identify the potentially cancerous lesion.

14. A multimodality imaging system for screening for epithelial cancer, comprising:
an endoscope defining a first channel for fluorescence imaging and a second channel for optical coherence tomography (OCT) imaging;
the endoscope comprising illumination fibers running coaxially with the first channel and the second channel, the illumination fibers coupled to a first source of radiation for the fluorescence imaging;
a distal end of the first channel configured to receive fluorescence radiation emitted by a dye of the cancer targeting agent and direct the fluorescence radiation to a first detector to acquire a fluorescence image of the tissue;
the second channel coupled to a second source for OCT imaging radiation, a distal end of the second channel configured to receive OCT radiation emitted by the tissue including a potentially cancerous lesion and direct the OCT radiation emitted to a second detector to acquire an OCT image of the tissue;
a processor coupled to the first detector and the second detector, the processor configured to identify the potentially cancerous lesion using the fluorescence imaging and image the potentially cancerous lesion for a cancer diagnosis of the tissue.

15. The multimodality imaging system of claim 14 further comprising a catheter configured to topically deliver the cancer targeting agent to the tissue.

16. The multimodality imaging system of claim 15 wherein the catheter is a third channel of the endoscope.

17. The multimodality imaging system of claim 14 wherein:
the illumination fibers are coupled to a third source of radiation for bright field imaging;
the distal end of the first channel is configured to receive radiation emitted by the tissue and direct the radiation to a third detector to acquire a bright field image of the tissue;
the processor is coupled to the third detector and is configured to co-register the bright field image and the fluorescence image of the tissue to identify the potentially cancerous lesion.

18. The multimodality imaging system of claim 14 wherein the cancer targeting agent is based on gold colloids adsorbed poly(epsilon-caprolactone) (Au-PCL) microparticles labeled with a near-infrared (NIR) dye and functionalized with an RGD peptide.

19. The multimodality imaging system of claim 14 wherein the cancer targeting agent is based on argenine-glycine-aspartic acid functionalized gold nanoparticles.

20. The multimodality imaging system of claim 14 wherein the cancer targeting agent is adapted to recognize αᵥβ₃ integrin receptors.

21. The multimodality imaging system of claim 14 wherein the cancer targeting agent is adapted to recognize EGF receptors.

22. A multimodality imaging system for screening for epithelial cancer, comprising:
a hand held probe including a first optic having a first surface configured (i) to direct first source radiation to tissue stained with a cancer targeting agent for fluorescence imaging, (ii) receive fluorescence radiation emitted by a dye of the cancer targeting agent in the tissue, and (iii) direct the fluorescence radiation to a first detector of the handheld probe to acquire a fluorescence image of the tissue;
a system of optics configured to raster scan, through a second surface of the first optic, OCT imaging radiation on the tissue including a potentially cancerous lesion, and direct OCT radiation emitted by the tissue to a second detector to acquire an OCT image of the tissue; and
a processor coupled to the first detector and the second detector, the processor configured to identify the potentially cancerous lesion using the fluorescence imaging and image the potentially cancerous lesion for a cancer diagnosis of the tissue.

23. The multimodality imaging system of claim 22 further comprising a catheter configured to topically deliver the cancer targeting agent to the tissue.

24. The multimodality imaging system of claim 22 wherein:
the first optic having a first surface is configured to direct third source radiation to tissue for bright field imaging, receive radiation emitted by the tissue, and direct the radiation to a third detector to acquire a bright field image of the tissue; and
the processor is coupled to the third detector and is configured to co-register the bright field image and the fluorescence image of the tissue to identify the potentially cancerous lesion.


SEQUENCE LISTING (1 sequence)

SEQ ID NO: 1 - protein, 15 aa, Artificial Sequence
  Description of Artificial Sequence Synthetic peptide
ACDCRGDCFCGGGGG